(19) United States
(12) Patent Application Publication
(10) Pub. No.: US 2023/0389905 A1
(43) Pub. Date: Dec. 7, 2023

(54) ORAL TESTING TOOL ASSEMBLY AND METHOD

(71) Applicant: David J. BOORD, North Canton, OH (US)

(72) Inventor: David J. BOORD, North Canton, OH (US)

(21) Appl. No.: 18/026,475

(22) PCT Filed: Sep. 16, 2021
(86) PCT No.: PCT/US2021/050610

§ 371 Date: Mar. 15, 2023

Related U.S. Application Data
(60) Provisional application No. 63/078,958, filed on Sep. 16, 2020.

(51) Int. Cl. A61B 10/02; G01N 33/543

(52) U.S. Cl. CPC A61B 10/0233; A61B 2010/0006; G01N 33/54388

(57) ABSTRACT

The present disclosure relates to an oral testing assembly and method comprising a head portion, an elongated handle attached to the head portion, and a testing strip configured to be positioned on a scraping surface of the head portion. A cover member including a detection window is configured to be attached to the head portion to support the testing strip to the head portion such that portions of the testing strip are exposed through a sample inlet and the detection window. The head portion and testing strip are arranged to collect a sample from the mouth or tongue of a patient to be received at the sample inlet and results displayed at the detection window. The head portion includes a scraping wall protruding from the scraping surface. The head portion may include a reservoir positioned along the scraping surface adjacent the scraping wall.

### CROSS REFERENCE TO RELATED APPLICATIONS

This application claims priority to and the benefit of U.S. Provisional patent application No. 63/078,958 filed on Sep. 16, 2020, entitled, ORAL TESTING TOOL ASSEMBLY AND METHOD, which is incorporated by reference in its entirety.

### FIELD OF THE DISCLOSURE

The present disclosure is generally related to a oral testing tool assembly and method for capturing a sample of cells or saliva, from the mouth of a patient and more particularly from a surface of a tongue of a patient that is configured to determine the existence of a viral load, such as SARS-CoV-2 within the collected sample.

### BACKGROUND

Currently, medical tests to determine the existence of a viral load, such as COVID-19 utilize enlarged swabs configured to be positioned within the nasal passage of an individual to procure cells or bio material for testing. This process is uncomfortable for the patient and questions have arisen as to the efficacy related to such procedures. Studies have indicated that saliva procured from the oral cavity from a patient is more sensitive for detecting a SARS-CoV-2 viral load than by use of enlarged nasopharyngeal swabs. These studies include Saliva is More Sensitive for SARS-CoV-2 detection in COVID-19 Patients than Nasophayrngeal Swabs, Anne L. Wyllie et al. Dated Apr. 22, 2020, as well as Is the Oral Cavity Relevant in SARS-CoV-2 Pandemic?, David Herrara et al. Dated Jun. 23, 2020.

As such, there is a need for an assembly, system and method to improve on the deficiencies of existing medical testing procedures that rely on nasal swabs or that could provide improved efficiencies for detection of a viral load for a variety of viruses that effect humans.

### SUMMARY

The present disclosure relates to an oral testing assembly and method of testing. In one embodiment, the oral testing assembly comprises a head portion, an elongated handle attached to the head portion, and a testing strip configured to be positioned on a scraping surface of the head portion. A cover member including a detection window is configured to be attached to the head portion to support the testing strip to the head portion such that portions of the testing strip are exposed through a sample inlet and the detection window. The head portion and testing strip are arranged to collect a sample from the mouth or tongue of a patient. The head portion includes a scraping wall protruding from the scraping surface of the head portion. The head portion may include a reservoir positioned along the scraping surface wherein the reservoir is defined within the scraping surface of the head portion and is adjacent the scraping wall. The sample inlet may be positioned in the reservoir. In an embodiment, the sample inlet may be positioned between the scraping wall and the cover member such that the sample inlet is spaced from the detection window such that a collected sample is configured to be received at the sample inlet to contact the testing strip and a test line and a control line are configured to be viewed or otherwise display test results through the detection window.

In one embodiment of the oral testing assembly, the testing strip is a lateral flow assay test strip having an elongated and rectangular shape. The scraping surface of the head portion may include a recessed area configured to receive and support the testing strip. The cover member may be configured to snap fit within the recessed area to support the testing strip in the recessed area. Additionally, the oral testing assembly may either be disposable or re-usable.

In a further embodiment, the cover member includes a body having a generally elongated and rectangular shape with a proximal end and a distal end, the proximal end defines a portion of the sample inlet. The body of the cover member may include a generally tapered thickness having a smaller thickness towards the proximal end and a larger thickness towards the distal end. The proximal end of the cover member may include an arcuate edge to define a portion of the sample inlet.

In another embodiment, the oral testing assembly may further comprise plurality of testing strips and aligned detection windows for detecting or displaying results for a plurality of different types of tests. A second testing strip may be positioned along the assembly wherein the cover member includes a second detection window such that portions of the second testing strip are exposed through a second sample inlet and the second detection window. The cover member may include a body having a generally elongated and rectangular shape with a proximal end and a distal end, the proximal end defines a portion of the sample inlet and having a protruding ridge adjacent the proximal end that defines a portion of a reservoir positioned along the scraping surface. In an embodiment, a surface protrusion may be positioned along at least one of the elongated handle and the cover member.

In another embodiment, provided is a method for testing a liquid sample for cutting disc for a food processor comprises providing an oral testing assembly head portion having a scraping surface and extending from an elongated handle, a testing strip configured to be positioned on the scraping surface of the head portion, and a cover member including a detection window wherein the cover member is configured to be attached to the head portion to support the testing strip to the head portion. A surface of a mouth of a patient may be scrapped with the scraping surface of the head portion. A sample may be directed to the sample inlet to be received by testing strip. Results may be provided on the test strip viewable through the detection window of the cover member. The oral testing assembly may include a plurality of aligned testing strips and detection windows such that a plurality of results may be read through the plurality of detection windows.

### BRIEF DESCRIPTION OF THE DRAWINGS

The disclosed assembly may be better understood by reference to the following detailed description taken in connection with the following illustrations, identified in FIGS. 1-12 attached hereto.

FIG. 1A is a perspective view of an embodiment of an oral testing tool assembly of the instant application;

FIG. 1B is an exploded perspective view of the oral testing tool assembly of FIG. 1A;

FIG. 2A is a perspective view of an embodiment of an oral testing tool assembly of the instant application;

FIG. 2B is an exploded perspective view of the oral testing tool assembly of FIG. 2A;

FIG. 3 is a front perspective view of the oral testing tool assembly of FIG. 2A without a testing strip of the instant application;

FIG. 4 is a rear perspective view of the oral testing tool assembly of FIG. 2A of the instant application;

FIG. 5A is a schematic view of a testing strip configured for use with the oral testing tool assembly of the instant application;

FIG. 5B is a schematic view of an embodiment of a testing strip illustrated with a saliva sample collected thereon;

FIG. 5C is a schematic view of an embodiment of a testing strip configured for use with the oral testing tool assembly of the instant application;

FIG. 5D is a schematic view of an embodiment of a testing strip configured for use with the oral testing tool assembly of the instant application;

FIG. 6A is a perspective view of an embodiment of an oral testing tool assembly of the instant application;

FIG. 6B is an exploded perspective view of the oral testing tool assembly of FIG. 6A;

FIG. 7A is a perspective view of an embodiment of an oral testing tool assembly of the instant application;

FIG. 7B is an exploded perspective view of the oral testing tool assembly of FIG. 7A;

FIG. 8 is an enlarged perspective view of a head of the oral testing tool assembly of FIG. 7A;

FIG. 9A is a rear perspective view of the oral testing tool assembly of FIG. 7A;

FIG. 9B is a rear exploded perspective view of the oral testing tool assembly of FIG. 7B;

FIG. 10 is a perspective view of another embodiment of an oral testing tool assembly of the instant application;

FIG. 11 is an exploded perspective view of the oral testing tool assembly of FIG. 10; and

FIG. 12 is a rear perspective view of the oral testing tool assembly of FIG. 10.

### DETAILED DESCRIPTION

Reference will now be made in detail to exemplary embodiments of the present disclosure, examples of which are illustrated in the accompanying Figures. It is to be understood that other embodiments may be utilized and structural and functional changes may be made without departing from the respective scope of the disclosure. Moreover, features of the various embodiments may be combined or altered without departing from the scope of the disclosure. As such, the following description is presented by way of illustration only and should not limit in any way the various alternatives and modifications that may be made to the illustrated embodiments and still be within the spirit and scope of the disclosure.

The present assembly described in this application involves embodiments that are related to prior embodiments related to tongue cleaning devices that have been commonly invented and disclosed by the following: U.S. Pat. No. 10,188,414 filed on Feb. 1, 2016 titled “Tongue Cleaning Device, U.S. patent application Ser. No. 15/935,758 filed on Mar. 26, 2018 titled “Portable Vacuum-Powered Tongue Cleaning Device,” U.S. patent application Ser. No. 16/261,237 on Jan. 29, 2019 titled “Tongue Cleaning Device,” and U.S. Provisional Patent Application No. 62/964,575 filed on Jan. 22, 2020 titled “Tongue Cleaning Device” each of which are incorporated by reference in their entireties.

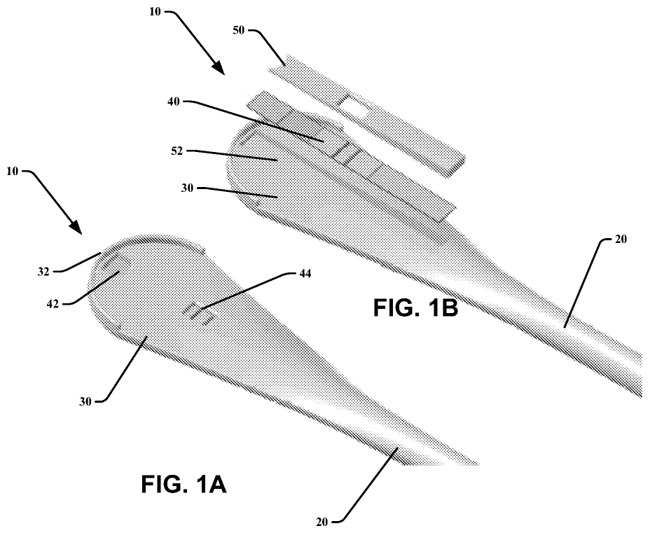

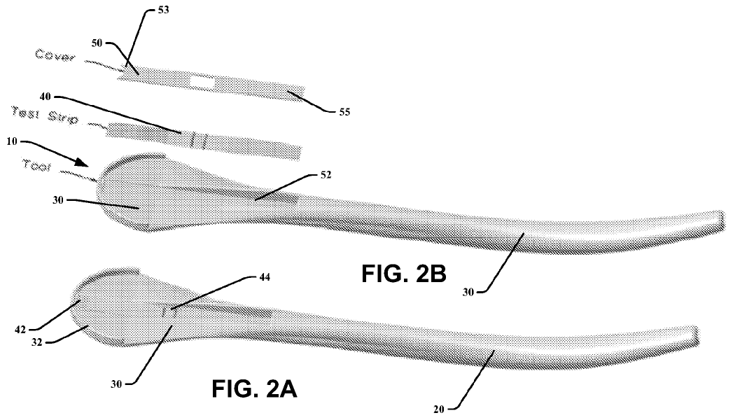

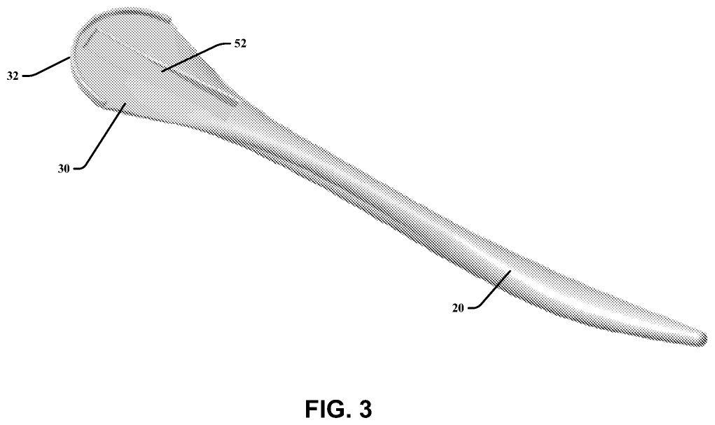

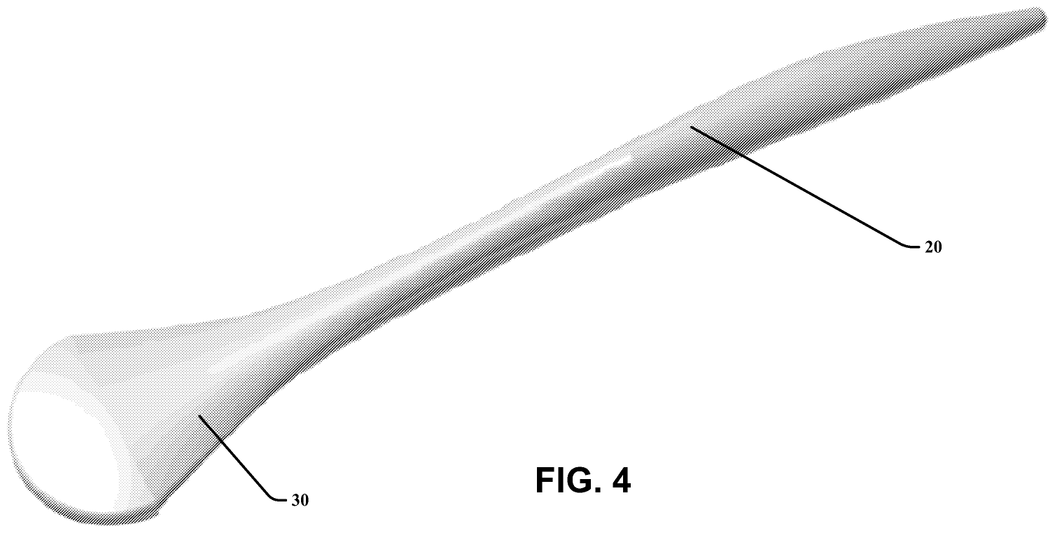

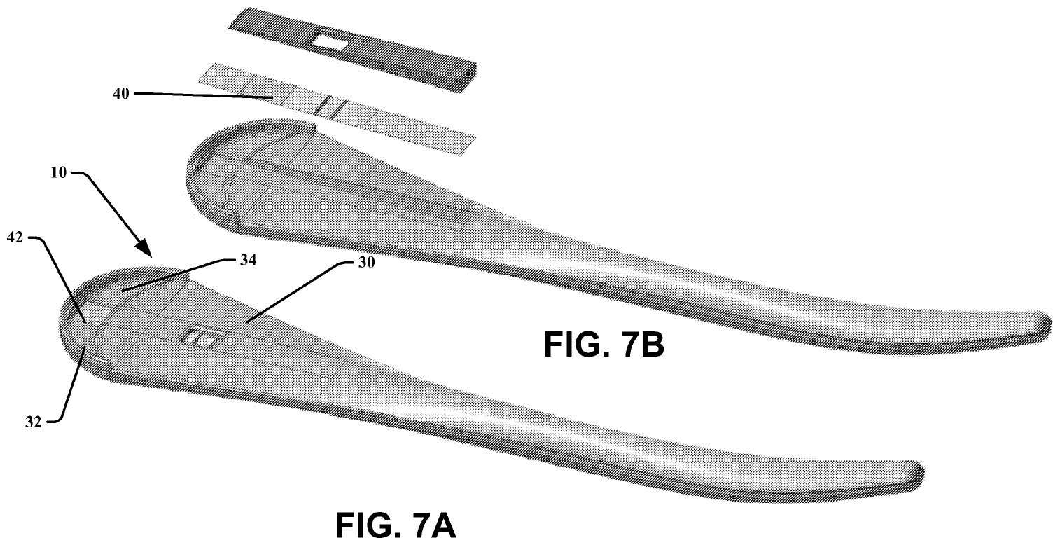

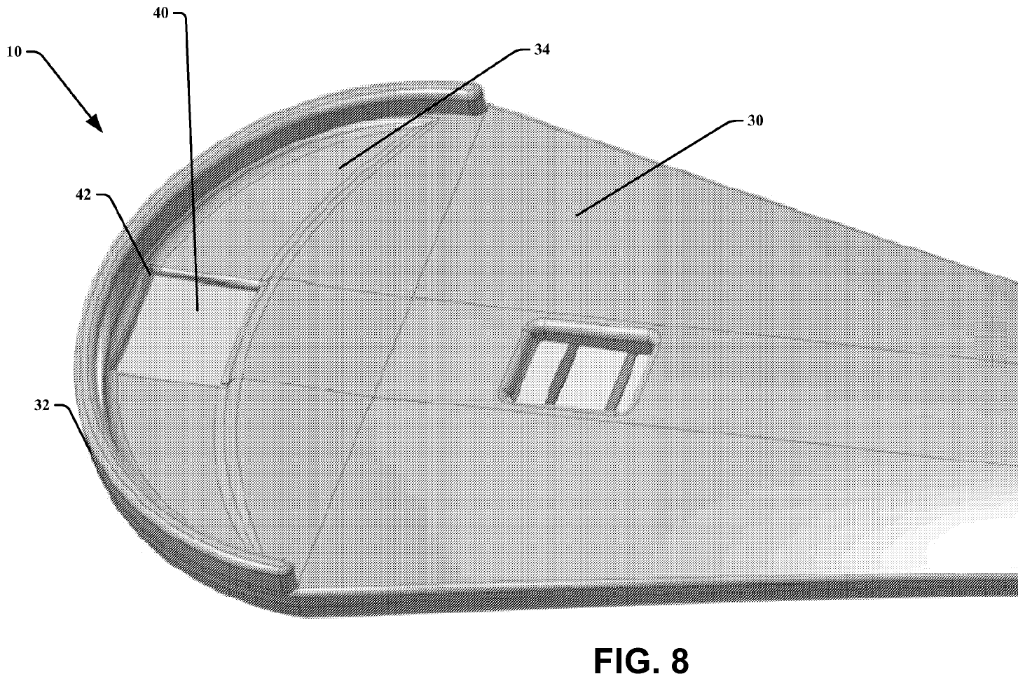

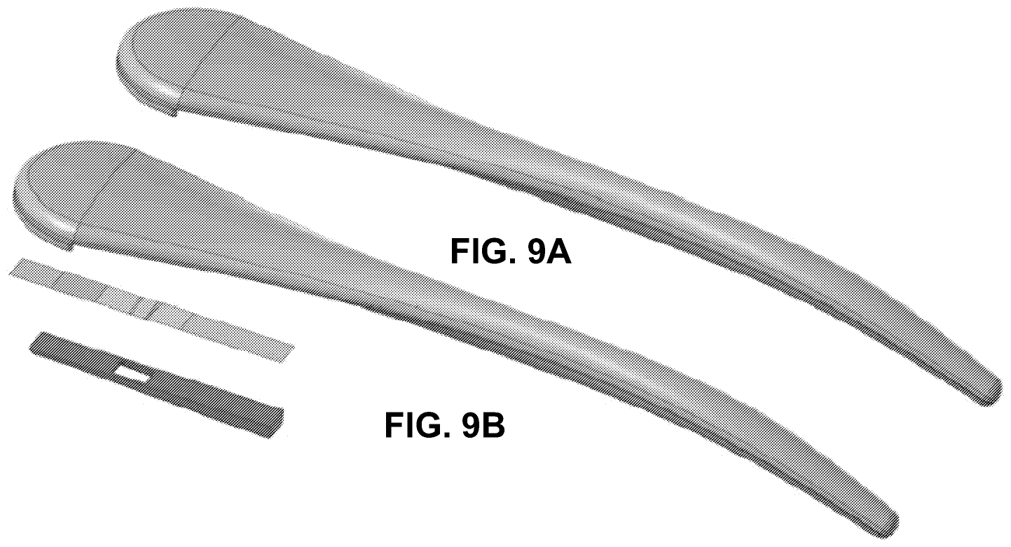

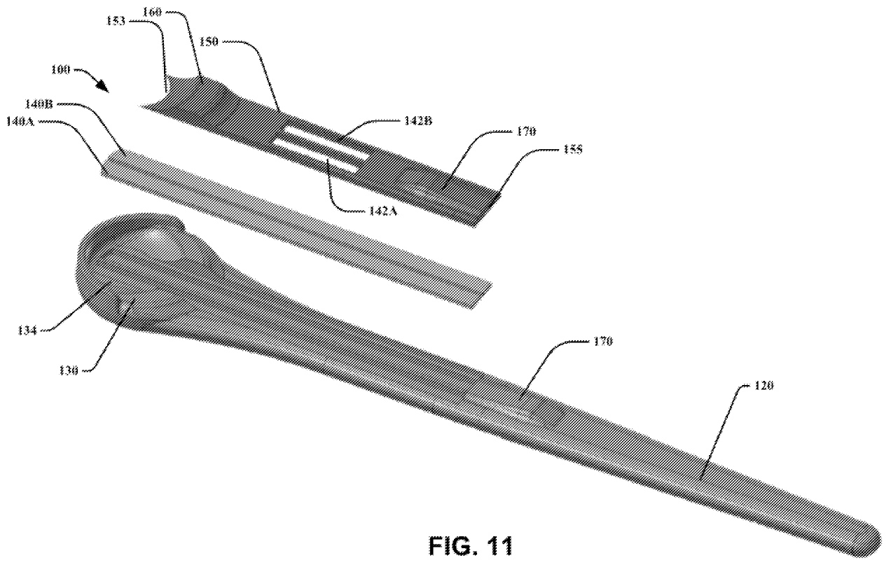

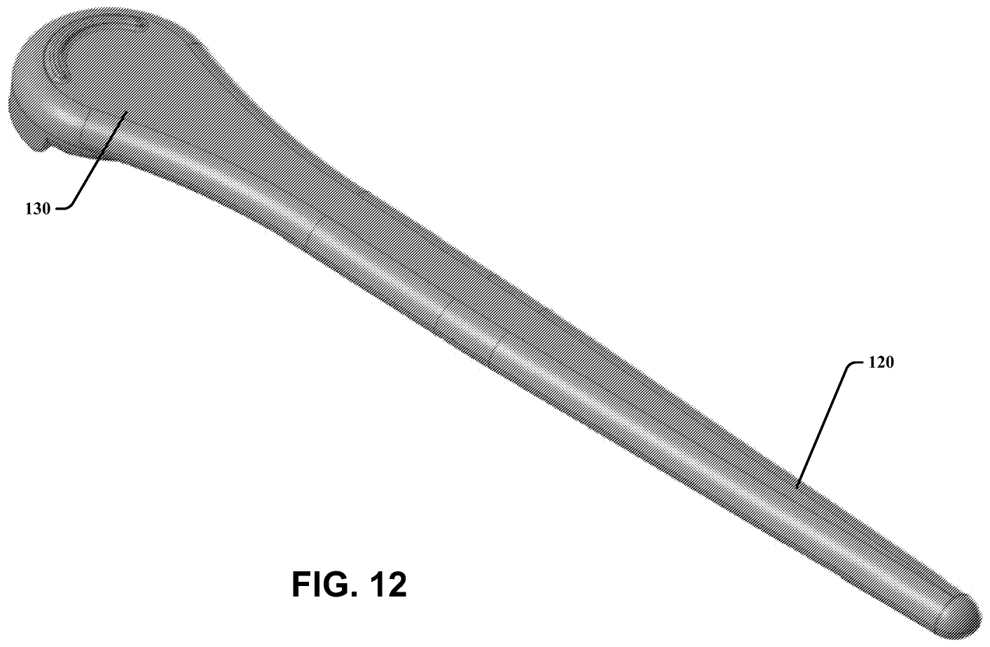

As illustrated by FIGS. 1-12, provided are embodiments of an oral test tool assembly 10 and method of testing for the presence of a viral load. The oral testing tool assembly 10 is configured to collect a sample from the tongue of a patient. The sample may be saliva, mucus, or other bio-matter or combination of such that may reside on the tongue of a patient. The oral test tool assembly 10 described may be configured to be used by a patient separately from medical professionals or directly with the aid of medical professionals. The oral test tool assembly 10 may be self administered by the patient at a home, office, or even vehicle without the need for assistance from a medical professional or for visiting a clinic, hospital or medical facility. Such self administration may be comparable to a home pregnancy test kit.

In one embodiment of FIGS. 1A, 1B, 2A, 2B, 3, and 4, the disclosed oral test tool assembly 10 includes an elongated handle 20 attached to a head portion 30. The head portion may include a testing strip 40 positioned on or within the head portion 30. The head portion 30 and testing strip 40 can be arranged to collect a liquid sample of saliva or bio-matter from the mouth or tongue of a patient and be configured to provide a rapid test for detecting the presence of a viral infection within the collected sample as will be discussed further. Such a viral load may be SARS-CoV-2 but may also be for other types of viruses known to be detectable by a lateral flow assay strip type of detection system for a liquid sample of saliva, mucus, or bio-matter that may be retrieved from the tongue or mouth of a patient, human or animal.

The oral test tool assembly 10 may be made of any particular material including may be made of a polymer, metal, wood, paper, alloy, cardboard and this disclosure is not limited. The head portion 30 may be configured with a scraping wall 32 having a generally arcuate shape and protruding from a bottom or scraping surface of the head portion 30. The scraping wall 32 may be configured to abut a surface within the mouth of a patient and glide against such surface to scrape or procure a sample from such surface of the patient.

The testing strip 40 may be placed within a recessed area 52 of the head portion 30 and retained therein by a cover member 50 to support the testing strip 40 to the head portion 30. However, the testing strip 40 may be placed or supported within the head portion 30 in any manner and this configuration is not limited. Notably, in some embodiment, the testing strip 40 and recessed area 52 may extend from the head portion 30 to the elongated handle 20. The cover member 50 may at least partially define a sample inlet 42 and a detection window 44 such that portions of the test strip 40 may be exposed to ambient through the sample inlet 42 and detection window 44. The sample inlet 42 may be defined by the cover member 50 and a portion of a surface of the head portion 30 such as a scraping surface or the scraping wall 32.

In one embodiment, the oral test tool assembly 10 may be configured to collect and house a liquid sample to position such sample within the sample inlet 42. The sample inlet 42 may be positioned adjacent to the scraping wall 32 along the head portion 30. The assembly may also be used to scrape or glide across a surface of the mouth such as the buccal cavity or an interior cheek or other inner mouth surface of the patient.

In this manner, the assembly 10 can scrape or otherwise procure a sample from the patient wherein such a sample may include stratified aquamous epithelium cells (ACE2 receptors) as well as saliva, mucus or other bio-matter to be collected or otherwise received within the sample inlet 42. Viral loads of particular clinical interest may be found within such samples. The assembly and method described herein may be used to detect the presence of a viral load of SARS-COV2 (COVID19) or the presence of any other type of diseases or viruses including but not limited to: dental caries (cavities), periodontal diseases, oral cancers, diabetes, cardiovascular disease, pancreatic cancer, breast cancer, lung cancer, prostate cancer, COVIDs, ebola, HIV, RSV (respiratory syncytial virus), pneumonia, influenza, AB disease (acid sphingomyelinase deficiency), etc. The sample collected may include the presence of epithelial cells having ACE2 host receptor cells.

In one embodiment, the testing strip 40 may be a lateral flow assay test strip. The testing assembly 10 may be configured to be cleaned and re-used while the testing strip 40 may be replaced after each use. Alternatively, the assembly 10 may be used and discarded after use. The testing strip 40 may be sold separately from the testing assembly 10 or may be packaged with a plurality of testing strips associated with one or more testing assemblies 10.

The testing assembly 10 may be economical, fast, and easy-to-use. It may provide for the detection of a viral load without expensive laboratory equipment. Such a testing strip may be available by DCN Diagnostics located in Carlsbad, California. The testing strips may be a nucleic acid lateral flow strip.

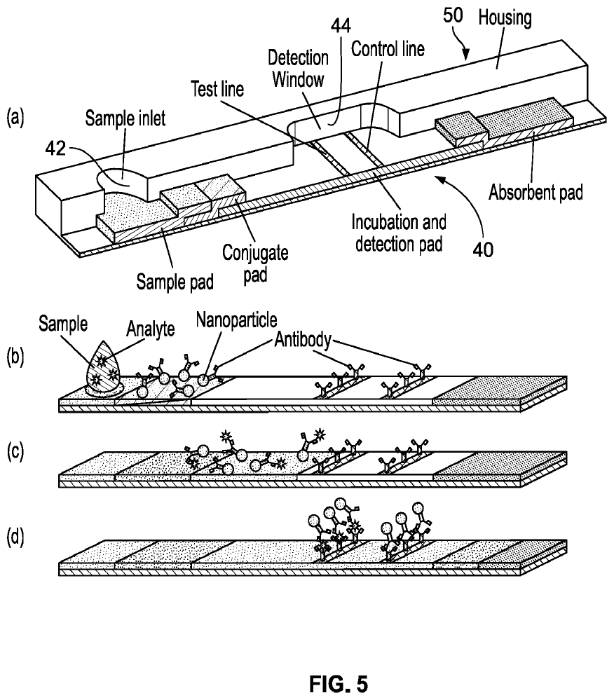

In this embodiment, as illustrated by FIGS. 5A-5D, the testing strip 40 may include a sample pad, a conjugate pad, an incubation and detection pad, and an absorbent pad. The sample pad may be positioned adjacent the sample inlet 42 to retain the sample as collected from the patient. The conjugate pad may separate the sample pad from the incubation and detection pad. The sample may be configured to transfer from the sample pad through the conjugate pad and to the incubation and detection pad. The flow of liquid sample may be performed by capillary action as the liquid sample translates from the sample inlet 42 along the length of the testing strip 40 within the body of the assembly 10.

The testing strip 40 may be configured to detect antibodies, nanoparticles, and analyte from the saliva, mucus, or bio-matter sample and present indicia along the incubation and detection pad that indicates either a positive or a negative result for the detection of a viral load. The indicia may be viewable and generally in alignment with the detection window 44 along the cover 50. The indicia may be in the form of at least one test line and a control line but any type of indicia may be contemplated in this application.

The testing strip may be considered a lateral assay test strip configured to detect a viral existence or other specific targets. The liquid sample may be presented within the sample inlet 42 and deposited onto the sample pad of the testing strip 40. In one embodiment, the sample pad would secure a prescribed antigen or other toxin therein. Such an antigen or toxin would induce an immune response in the production of antibodies. The sample pad may also be treated, configured, or otherwise adjusted to the target sample properties.

At this point, through capillary action, the sample containing the specific target antigen or toxin from said sample would flow to the conjugate pad section of the testing strip 40. The conjugate pad may include specific antibodies pertaining to the type of viral load or infection that the testing strip is desired to detect. As the sample flows through the conjugate pad of the testing strip, the sample become rehydrated within the testing strip to allow the sample to bind with matching colored labeled antibodies.

The sample continues to flow through the conjugate pad to the incubation and detection pad. In one embodiment, the incubation and detection pad may be a nitrocellulose membrane that includes test and control lines. The test line and control lines may include capture reagents. The incubation and detection pad may allow the sample or molecules from said sample to bind to a color labeled antibody. Together, the sample and color labeled antibodies flow up the device or along the testing strip 40. Any unbound antibodies may bind to the target toxins applied at the test line to form a visible line. In an embodiment, the less intense the test line or indicia viewable through the detection window 44, the more of a toxin or viral load is present in the sample. Excess colored labeled antibodies bind to the anispices applied to a control line. Notably, the control line and test line may be separate lines along the testing strip and are configured to be viewable through the detection window 44. The control line becomes viewable regardless of the presence or absence of the target molecules in the sample and assures that the testing assembly 10 is working correctly. The above results in a positive test result.

In contrast, for a negative test result, a sample is applied to the testing strip 40 through the sample inlet 42. The sample combines with colored labeled antibodies. However, as no target molecules, target antigen or toxins are present, colored label antibodies continue to flow up the device or along the testing strip 40 within the assembly 10 in an unbound manner. Unbound antibodies bind to the target toxins applied at the test line. In an embodiment, the more visually intense the test line through the detection window 44, the less of a toxin or viral load is present in the sample. As in a positive test outcome, excess colored labeled antibodies bind to the anitspices applied at the control line. This action occurs regardless of the presence or absence of the target molecules in the sample and assures the testing assembly 10 and is working correctly. In an embodiment, a result of no binding of the sample target to the antibodies a visual test results in a negative test result.

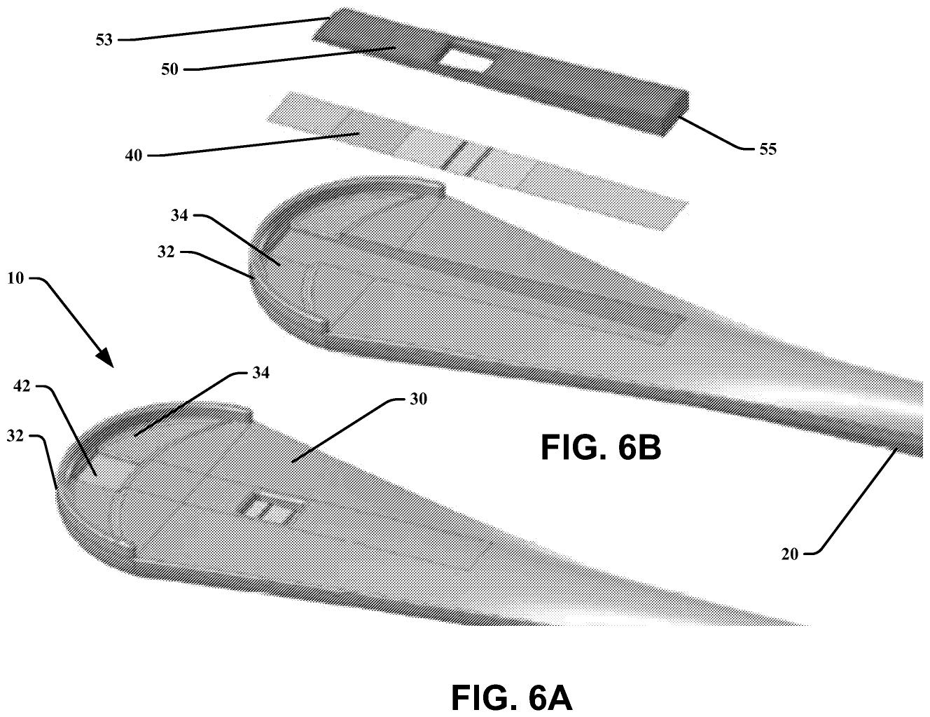

FIGS. 6A, 6B, 7A, 7B, 8, 9A, and 9B illustrate another embodiment of the oral testing assembly 10 having a head portion 30 with an arcuate shape and an elongated handle 20 wherein the assembly has a generally flexible configuration. These figures illustrate yet another embodiment of the oral testing assembly 10 wherein the head portion 30 includes a reservoir 34 positioned along a scraping surface thereon. The reservoir 34 may be defined within the surface of the head portion 30 and be adjacent the scraping wall 32. The sample inlet 42 may be located within the reservoir 34 and be configured such that as the oral testing assembly 10 abuts against the surface within a mouth of a patient, sample bio matter such as saliva or mucus may be scraped therefrom by the scraping wall 32 and be retained within the reservoir 34. At least a portion of the collected sample may enter the sample inlet 42 to make sufficient contact with the testing strip 40 therein.

The cover member 50 includes a body having a generally elongated and rectangular shape with a proximal end 53 and a distal end 55, the proximal end may define a portion of the sample inlet 42 that can otherwise be defined by the scraping surface and scraping wall 32. The body of the cover member 50 may include a generally tapered thickness having a smaller thickness towards the proximal end 53 and a larger thickness towards the distal end 55. The proximal end of the cover member may include an arcuate edge or may be a generally straight and angled edge to define a portion of the sample inlet 42.

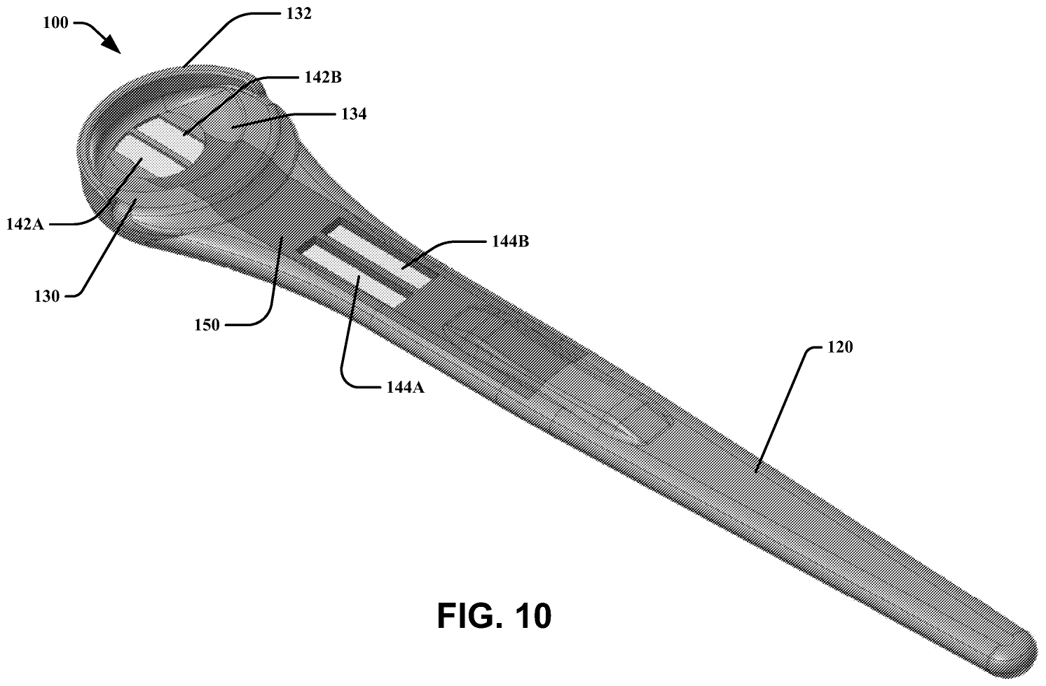

FIGS. 10, 11 and 12 illustrate another embodiment of the oral testing assembly 100. In this embodiment, the assembly includes a head portion 130, a cover member 150, and an elongated handle 120 but may further comprise a plurality of testing strips 140A, 140B and a plurality of sample inlets 142A, 142B, and detection windows 144A, 144B for collecting samples and displaying results for a plurality of different types of tests. This embodiment may allow a single sample be collected to test for as many different types of viruses as there are testing strips. Notably, this disclosure contemplates that any number of testing strips may be used and FIGS. 10-12 only illustrate the use of 2 testing strips. However, 3, 4, 5 or 6 or more are certainly contemplated herein.

The first and second testing strips 140A, 140B may be positioned along the assembly wherein a first recessed area 152A is aligned with but separated from a second recessed area 152B wherein each recessed area is configured to support a separate testing strip therein. In an embodiment, the recessed areas are configured to prevent migration of liquid sample from one testing strip to the other. The cover member 150 includes first and second detection windows 144A, 144B such that portions of the first and second testing strips 140A, 140B are exposed through respective first and second sample inlets 142A, 142B and the first and second detection windows 144A, 144B. The cover member 150 may include a body having a generally elongated and rectangular shape with a proximal end 153 and a distal end 155, the proximal end defines a portion of the first and second sample inlets 142A, 142B. A protruding ridge 160 may be positioned along the cover member 150 to assist with defining a shape of a reservoir 134 formed in the head portion 130. The protruding ridge 160 may be adjacent the proximal end 153 that defines a portion of the reservoir 134 positioned along the scraping surface. In an embodiment, a surface protrusion 170 may be positioned along at least one of the elongated handle 120 and the cover member 150 or may extend from the cover member 150 to the elongated handle 120 to allow for alignment and attachment of the cover member 150 with the head portion and elongated handle.

In an embodiment, the testing assembly 10 has been found to provide a specificity of about 95%. Further, the testing assembly 10 may include a sensitivity of about 95%. The testing assembly may be configured to provide detection or test results in about 5 minutes or less after procuring the sample from the patient.

The testing assembly is configured to be performed at home, school, work, nursing home, hospital, athletic event, or any type of social gathering. Further, the entire testing assembly may have a low costs and may be easy to procure and assemble, can be self-administered or health care provider can also provider test. The testing assembly may include no moving parts and may be administered with a single hand.

Although, the instant disclosure is particularly related to uncovering the existence of the COVID-19 virus in samples collected from patients, the instant disclosure is not limited as such and may be utilized to test for a variety of illnesses that may exist including those determined by samples collected from the mouths or tongues of a patient.

Although the embodiments of the present disclosure have been illustrated in the accompanying drawings and described in the foregoing detailed description, it is to be understood that the present disclosure is not to be limited to just the embodiments disclosed, but that the disclosure described herein is capable of numerous rearrangements, modifications and substitutions without departing from the scope of the claims hereafter. The scope of this disclosure is intended to include all modifications and alterations insofar as they come within the scope of the disclosure or the equivalent thereof.

What is claimed is:

1. An oral testing assembly comprising:
a head portion;
an elongated handle attached to the head portion;
a testing strip configured to be positioned on a scraping surface of the head portion; and
a cover member including a detection window wherein the cover member is configured to be attached to the head portion to support the testing strip to the head portion such that portions of the testing strip are exposed through a sample inlet and the detection window;
wherein the head portion and testing strip are arranged to collect a sample from the mouth or tongue of a patient.

2. The oral testing assembly of claim 1, wherein the head portion includes a scraping wall protruding from the scraping surface of the head portion.

3. The oral testing assembly of claim 2, wherein the head portion includes a reservoir positioned along the scraping surface wherein the reservoir is defined within the scraping surface of the head portion and is adjacent the scraping wall.

4. The oral testing assembly of claim 3, wherein the sample inlet is positioned in the reservoir.

5. The oral testing assembly of claim 2, wherein the sample inlet is positioned between the scraping wall and the cover member such that the sample inlet is spaced from the detection window.

6. The oral testing assembly of claim 1, wherein a sample is configured to be received at the sample inlet to contact the testing strip and a test line and a control liner are configured to be viewed through the detection window.

7. The oral testing assembly of claim 1, wherein the testing strip is a lateral flow assay test strip having an elongated and rectangular shape.

8. The oral testing assembly of claim 1, wherein the scraping surface of the head portion includes a recessed area configured to receive and support the testing strip

9. The oral testing assembly of claim 8, wherein the cover member is configured to snap fit within the recessed area to support the testing strip in the recessed area.

10. The oral testing assembly of claim 1, wherein the oral testing assembly is disposable.

11. The oral testing assembly of claim 1, wherein the oral testing assembly is re-usable.

12. The oral testing assembly of claim 1, wherein the cover member includes a body having a generally elongated and rectangular shape with a proximal end and a distal end, the proximal end defines a portion of the sample inlet.

13. The oral testing assembly of claim 12, wherein the body of the cover member includes a generally tapered thickness having a smaller thickness towards the proximal end and a larger thickness towards the distal end.

14. The oral testing assembly of claim 12, wherein the proximal end of the cover member includes an arcuate edge to define a portion of the sample inlet.

15. The oral testing assembly of claim 1, further comprising a second testing strip wherein the cover member includes a second detection window such that portions of the second testing strip are exposed through a second sample inlet and the second detection window.

16. The oral testing assembly of claim 15, wherein the cover member includes a body having a generally elongated and rectangular shape with a proximal end and a distal end, the proximal end defines a portion of the sample inlet and having a protruding ridge adjacent the proximal end that defines a portion of a reservoir positioned along the along the scraping surface.

17. The oral testing assembly of claim 15, further comprising a surface protrusion positioned along at least one of the elongated handle and the cover member.

18. A method for testing a liquid sample for cutting disc for a food processor comprising:
providing an oral testing assembly head portion having a scraping surface and extending from an elongated handle, a testing strip configured to be positioned on the scraping surface of the head portion, and a cover member including a detection window wherein the cover member is configured to be attached to the head portion to support the testing strip to the head portion;
scraping a surface of a mouth of a patient with the scraping surface of the head portion; directing a sample to the sample inlet to be received by testing strip; and reading results on the test strip through the detection window of the cover member.

19. The method of claim 18, wherein the oral testing assembly includes a plurality of aligned testing strips and detection windows such that a plurality of results may be read through the plurality of detection windows.

20. The method of claim 18 wherein the head portion includes a scraping wall protruding from the scraping surface of the head portion and a reservoir in the scraping surface.
